(19) United States
(12) Patent Application Publication
(10) Pub. No.: US 2018/0155749 A1
(43) Pub. Date: Jun. 7, 2018

(54) PROCESS FOR PRODUCING ACYL AMINO ACIDS EMPLOYING LIPASES

(71) Applicants: Katrin GRAMMANN, Oer-Erkenschwick (DE); Hans-Georg HENNEMANN, Bedburg (DE); Nicole DECKER, Bochum (DE); Nicole KIRCHNER, Recklinghausen (DE); Liv REINECKE, Essen (DE)

(72) Inventors: Katrin GRAMMANN, Oer-Erkenschwick (DE); Hans-Georg HENNEMANN, Bedburg (DE); Nicole DECKER, Bochum (DE); Nicole KIRCHNER, Recklinghausen (DE); Liv REINECKE, Essen (DE)

(21) Appl. No.: 15/571,719

(22) PCT Filed: Apr. 28, 2016
(86) PCT No.: PCT/EP2016/059456

§ 371 Date: Nov. 3, 2017

(30) Foreign Application Priority Data
May 5, 2015 (EP) 15166355.6

(51) Int. Cl. C12P 13/04; C12N 9/20; C12N 9/10; C12N 9/00

(52) U.S. Cl. CPC C12P 13/04; C12N 9/20; C12Y 602/01003; C12N 9/93; C12N 9/1029

(57) ABSTRACT

There is provided a microbial cell for producing at least one acyl amino acid, wherein the cell is genetically modified to comprise:
a first genetic mutation that increases the expression relative to a wild type cell of at least one lipase (EC 3.1.1) (E₁) capable of hydrolysing at least one glyceride to at least one fatty acid wherein the glyceride is a triglyceride; and
a second and a third genetic mutation that increases the expression relative to a wild type cell of:
(i) an amino acid-N-acyl-transferase (EC 2.3.1) (E₂), and
(ii) an acyl-CoA synthetase (EC 6.2.1.3) (E₃) respectively
that enables the cell to convert the fatty acid to at least one acyl amino acid and
wherein the cell has a reduced fatty acid degradation capacity.

### FIELD OF THE INVENTION

The present invention relates to biotechnological methods and cells for producing at least one acyl amino acid from oils.

### BACKGROUND OF THE INVENTION

Acyl amino acids are a class of surface-active agents with a variety of uses, for example as detergents for washing purposes, emulsifiers in food products and as essential ingredients in various personal care products such as shampoos, soaps, moisturizing agents and the like. In addition to having both hydrophobic and hydrophilic regions, a prerequisite for use as a surfactant, requires these surfactants to be made of naturally occurring molecules. These naturally occurring molecules include amino acids and fatty acids, which are not only non-hazardous and environmentally acceptable but may be readily produced at a large scale using inexpensive biological raw materials. Acyl amino acids may also be used in pharmacological research as neuromodulators and/or probes for new drug targets.

Traditionally acyl amino acids have been produced at an industrial scale starting with materials derived from tropical oils. More specifically, activated fatty acids provided in the form of acid chlorides have been used to acylate amino acids in an aqueous alkaline medium as described in GB 1 483 500. Shortcomings of such approaches include the need to add hazardous chemicals such as sulphuric acid or anhydrides thereof. Other synthetic approaches are associated with the accumulation of by-products such as chloride salts which have undesirable effects on surfactancy. For example, Schotten-Baumann synthesis is an example of a synthesis route that is commonly used for the production of acyl amino acids. However, this method has the disadvantage that chlorinated fatty acids are used in the method and these fatty acids are synthesized via toxic chemicals like phosgene or PCL3.

A range of biotechnological routes towards production of acyl amino acids has been described. However, none of them is adequate for the commercial large-scale production of acyl amino acids owing to low yields, insufficient purities and the need for multi-step purification procedures. In particular, only a small proportion of the carbon substrates fed to biotechnologically useful organisms is actually converted to acyl amino acids, whilst much of these substrates is consumed by reactions of the primary metabolism.

Another problem associated with biotechnological routes is the fact that a mixture of products is obtained and thus the composition is difficult to control. More specifically, a range of fatty acids may be converted to acyl amino acids, even though production of a single adduct may be desirable. This also leads to foaming which makes controlling the reaction difficult. Since the mixture comprises compounds highly related in terms of chemical structure, purifying or at least enriching a single component in an efficient and straightforward manner is usually beyond technical feasibility.

Accordingly, there is a need in the art for a means of producing acyl amino acids more efficiently and at lower costs.

### DESCRIPTION OF THE INVENTION

The present invention attempts to solve the problems above by providing at least one cell and/or method of producing at least one acyl amino acid from any naturally existing oil. This is advantageous as it allows for the use of naturally occurring oil instead of fatty acids. An additional hydrolysis step for the preparation of free fatty acids may thus be avoided. This allows the process of acyl amino acid production to be sped up and also saves cost. Further, the use of oils as a substrate for acyl amino acid production may be advantageous as the oil results in a less foamy effect and therefore less or no anti foaming agent needs to be used in the method according to any aspect of the present invention.

According to one aspect of the present invention there is provided a microbial cell for producing at least one acyl amino acid, wherein the cell is genetically modified to comprise:
a first genetic mutation that increases the expression relative to a wild type cell of at least one lipase (EC 3.1.1) (E₁) capable of hydrolysing at least one glyceride to at least one fatty acid; and
a second and a third genetic mutation that increases the expression relative to a wild type cell of:
(i) an amino acid-N-acyl-transferase (EC 2.3.1) (E₂), and
(ii) an acyl-CoA synthetase (EC 6.2.1.3) (E₃) respectively
that enables the cell to convert the fatty acid to at least one acyl amino acid and
wherein the cell has a reduced fatty acid degradation capacity.

The cell according to any aspect of the present invention may be capable of converting at least one natural oil to free fatty acid(s) and the cell may further comprise a second and a third genetic mutation that enables the cell to produce at least one acyl amino acid from the fatty acid. In particular, the first genetic mutation may enable to cell to convert a naturally occurring and cheap source of fats to a fatty acid that may then be used as a suitable raw material for the production of more useful higher organic compounds such as acyl amino acids. More in particular, the first genetic mutation may be of at least one lipase that converts glycerides that are found in natural oils to fatty acids. The glyceride may be a monoglyceride, diglyceride or triglyceride. In particular, the glyceride used according to any aspect of the present invention may be a triglyceride.

The terms “natural oils,” may refer to oils derived from plants or animal sources. The term “natural oil” includes natural oil derivatives. Natural oils may also include modified plant or animal sources (e.g., genetically modified plant or animal sources). Examples of natural oils include, but are not limited to, vegetable oils, algae oils, fish oils, animal fats, tall oils, derivatives of these oils, combinations of any of these oils, and the like. Non-limiting examples of vegetable oils include canola oil, rapeseed oil, coconut oil, corn oil, cottonseed oil, olive oil, palm oil, peanut oil, safflower oil, sesame oil, soybean oil, sunflower oil, linseed oil, palm kernel oil, tung oil, jatropha oil, mustard oil, pennycress oil, camelina oil, and castor oil. Natural oils may also include animal fats such as lard, tallow, poultry fat, yellow grease, and fish oil. Tall oils are by-products of wood pulp manufacture. Any one of these natural oils may be used as a source of glycerides according to any aspect of the present invention.

Lipases are enzymes that catalyse hydrolysis reactions to decompose oils into free fatty acids and glycerol. Various types of animals, plants and microorganisms are known to have lipases. Differences in the activity of lipases occur due to bonding position within the glyceride, carbon chain length of the fatty acid, number of double bonds, and the like. It is thus possible to selectively hydrolyse fatty acids using such lipases, and as a result, it becomes possible to concentrate a specific fatty acid within the glyceride fraction. For example, when a lipase produced by a kind of the genus Candida is used, it is known that hydrolysis reaction of fish oil results in concentration of highly unsaturated fatty acids, such as docosahexaenoic acid, in the undecomposed glyceride fraction. Similarly, the suitable lipase may be selected to be used to hydrolyse the relevant oils to the desired fatty acid and glycerol. In one example, the lipase used according to any aspect of the present invention may be derived from a microorganism selected from the group consisting of: Candida cylindracea, Alcaligenes sp., Burkholderia cepacia, Pseudomonas fluorescens, Thermomyces lanuginosus, Rhizomucor miehei, and Pseudomonas sp. In particular, the lipase may be from Thermomyces lanuginosus. Such lipases include lipases obtained from microorganisms belonging to Alcaligenes sp. (Lipase QLM, Lipase QLC, Lipase PL, all produced by Meito Sangyo Co., Ltd.), lipases obtained from microorganisms belonging to Burkholderia cepacia (Lipase PS, produced by Amano Enzyme Inc.), lipases obtained from microorganisms belonging to Pseudomonas fluorescens (Lipase AK, produced by Amano Enzyme Inc.), lipases obtained from microorganisms belonging to Thermomyces lanuginosus (Lipozyme TLIM, produced by Novozymes), or the like. Lipases derived from Candia rugose, Rhizomucor miehei, Pseudomonas sp. and the like. Poulsen at al. show the effectiveness of a variety of lipases such as from Fusarium solani pisi (cutinase), Rhizomucor miehei, Pseudomonas cepacia, and Humicola lanuginose. In particular, the lipase may be from Thermomyces lanuginosus. Lipases from Thermomyces lanuginosus have been shown to be effective in Yan et al. All of these lipases are commercially marketed and may be readily obtained. As may be required, these lipases may be immobilized prior to use.

More in particular, the lipase (E₁) used according to any aspect of the present invention may comprise a sequence of 50%, 55%, 60%, 65%, 70%, 75%, 80%, 85%, 90%, 95%, 100% sequence identity with SEQ ID NO:4.

The second and third genetic mutation in the cell may be in at least one amino acid-N-acyl-transferase (E₂) and (ii) acyl-CoA synthetase (E₃) respectively. In another example, the second genetic mutation may result in increased activity relative to the wild type cell of glycine N-acyl transferase (E₂ₐ). In yet another example, the cell according to any aspect of the present invention may be genetically modified to have a mutation in an enzyme that is involved in a lipase (E₁) and a mutation in an enzyme that may result in increased activity relative to the wild type cell of glycine-N-acyl transferase (E₂ₐ).

According to any aspect of the present invention, the second and third genetic mutation may result in the cell having increased expression of (i) amino acid-N-acyl-transferase (E₂) and (ii) acyl-CoA synthetase (E₃) respectively. The combination of amino acid-N-acyl transferase and an acyl-CoA synthetase, expressed according to any aspect of the present invention may be used to convert a variety of fatty acids and/or amino acids including a mixture comprising unsaturated and saturated fatty acids, to acyl amino acids. In particular, the amino acid-N-acyl transferases may be used to convert short unsaturated fatty acids such as lauroleic acid to an acyl amino acid. More in particular, the amino acid-N-acyl-transferase may be capable of converting a variety of fatty acids including short unsaturated fatty acids such as lauroleic acid to an acyl amino acid with an increase in the yields of acyl amino acids produced. Further, the composition of acyl amino acids produced in a cell, more specifically the length of fatty acids incorporated into such acyl amino acids, may be controlled by introducing into the cell one or more specific acyl-CoA thioesterases or altering the expression of one or more acyl-CoA thioesterases endogenously expressed by the cell.

The cell according to any aspect of the present invention may comprise a further genetic mutation that enables the cell to have increased expression of acyl-CoA thioesterase (E₅). The term “acyl-CoA thioesterase”, as used herein, refers to an enzyme capable of hydrolysing acyl-CoA. In particular, the acyl-CoA thioesterase comprises a sequence selected from the group consisting of AEM72521.1 and AAC49180.1 or a variant thereof. The nucleotide sequence of E₅ may be SEQ ID NO:3. The activity of acyl-CoA thioesterase may be assayed using various assays described in the state of the art. Briefly, the reaction of Ellman's reagent, which reacts with free thiol groups associated with CoASH formed upon hydrolysis of acyl-CoA may be detected by spectophotometrically monitoring absorbance at 412 nm.

The term “amino acid-N-acyl transferase”, as used herein, refers to an enzyme capable of catalysing the conversion of acyl-CoA, for example the CoA ester of an acid, and an amino acid, for example a proteinogenic amino acid, for example glycine, glutamic acid, and/or any other amino acid to an acyl amino acid. Suitable amino acid-N-acyl transferases have been described in the prior art, for example in Waluk, D. P., Schultz, N., and Hunt, M. C. (2010). In particular, the amino acid sequence of amino acid-N-acyl transferase may be selected from the group consisting of SEQ ID NO:1, NP_659453.3 NP_001010904.1, XP_001147054.1, AAH16789.1, AA073139.1, XP_003275392.1, XP_002755356.1, XP_003920208.1, XP_004051278.1, XP_006147456.1, XP_006214970.1, XP_003801413.1, XP_006189704.1, XP_003993512.1, XP_005862181.1, XP_007092708.1, XP_006772167.1, XP_006091892.1, XP_005660936.1, XP_005911029.1, NP_001178259.1, XP_004016547.1, XP_005954684.1, ELR45061.1, XP_005690354.1, XP_004409352.1, XP_007519553.1, XP_004777729.1, XP_005660935.1, XP_004824058.1, XP_006068141.1, XP_006900486.1, XP_007497585.1, XP_002821801.2, XP_007497583.1, XP_003774260.1, XP_001377648.2, XP_003909843.1, XP_003801448.1, XP_001091958.1, XP_002821798.1, XP_005577840.1, XP_001092197.1, NP_001207423.1, NP_001207425.1, XP_003954287.1, NP_001271595.1, XP_003909848.1, XP_004087850.1, XP_004051279.1, XP_003920209.1, XP_005577835.1, XP_003774402.1, XP_003909846.1, XP_004389401.1, XP_002821802.1, XP_003774401.1, XP_007497581.1, EHH21814.1, XP_003909845.1, XP_005577839.1, XP_003774403.1, XP_001092427.1, XP_003275395.2, NP_542392.2, XP_001147271.1, XP_005577837.1, XP_003826420.1, XP_004051281.1, XP_001147649.2, XP_003826678.1, XP_{—} 003909847.1, XP_004682812.1, XP_004682811.1, XP_003734315.1, XP_004715052.1, BAG62195.1, XP_003777804.1, XP_003909849.1, XP_001092316.2, XP_006167891.1, XP_540580.2, XP_001512426.1, EAW73833.1, XP_003464217.1, XP_007519551.1, XP_003774037.1, XP_005954680.1, XP_003801411.1, NP_803479.1, XP_004437460.1, XP_006875830.1, XP_004328969.1, XP_004264206.1, XP_004683490.1, XP_004777683.1, XP_005954681.1, XP_003480745.1, XP_004777682.1, XP_004878093.1, XP_007519550.1, XP_003421399.1, EHH53167.1, XP_006172214.1, XP_003993453.1, AAI12537.1, XP_006189705.1, Q2KIR7.2, XP_003421465.1, NP_001009648.1, XP_003464328.1, XP_001504745.1, ELV11036.1, XP_005690351.1, XP_005216632.1, EPY77465.1, XP_005690352.1, XP_004016544.1, XP_001498276.2, XP_004264205.1, XP_005690353.1, XP_005954683.1, XP_004667759.1, XP_004479306.1, XP_004645843.1, XP_004016543.1, XP_002928268.1, XP_006091904.1, XP_005331614.1, XP_007196549.1, XP_007092705.1, XP_004620532.1, XP_004869789.1, EHA98800.1, XP_004016545.1, XP_004479307.1, XP_004093105.1, NP_001095518.1, XP_005408101.1, XP_004409350.1, XP_001498290.1, XP_006056693.1, XP_005216639.1, XP_007455745.1, XP_005352049.1, XP_004328970.1, XP_002709220.1, XP_004878092.1, XP_007196553.1, XP_006996816.1, XP_005331615.1, XP_006772157.1, XP_007196552.1, XP_004016546.1, XP_007628721.1, NP_803452.1, XP_004479304.1, DAA21601.1, XP_003920207.1, XP_006091906.1, XP_003464227.1, XP_006091903.1, XP_006189706.1, XP_007455744.1, XP_004585544.1, XP_003801410.1, XP_007124812.1, XP_006900488.1, XP_004777680.1, XP_005907436.1, XP_004389356.1, XP_007124811.1, XP_005660937.1, XP_007628724.1, XP_003513512.1, XP_004437813.1, XP_007628723.1, ERE78858.1, EPQ15380.1, XP_005862178.1, XP_005878672.1, XP_540581.1, XP_002928267.1, XP_004645845.1, EPQ05184.1, XP_003513511.1, XP_006214972.1, XP_007196545.1, XP_007196547.1, XP_006772160.1, XP_003801409.1, NP_001119750.1, XP_003801412.1, XP_006772159.1, EAW73832.1, XP_006091897.1, XP_006772163.1, XP_006091898.1, XP_005408105.1, XP_006900487.1, XP_003993454.1, XP_003122754.3, XP_007455746.1, XP_005331618.1, XP_004585337.1, XP_005063305.1, XP_006091895.1, XP_006772156.1, XP_004051276.1, XP_004683488.1, NP_666047.1, NP_001013784.2, XP_006996815.1, XP_006996821.1, XP_006091893.1, XP_006173036.1, XP_006214971.1, EPY89845.1, XP_{—} 003826423.1, NP_964011.2, XP_007092707.1, XP_005063858.1, BAL43174.1, XP_001161154.2, XP_007124813.1, NP_083826.1 XP_003464239.1, XP_003275394.1, ELK23978.1, XP_004878097.1, XP_004878098.1, XP_004437459.1, XP_004264204.1, XP_004409351.1, XP_005352047.1, Q5RFP0.1, XP_005408107.1, XP_007659164.1, XP_003909852.1, XP_002755355.1, NP_001126806.1, AAP92593.1, NP_001244199.1, BAA34427.1, XP_005063859.1, NP_599157.2, XP_004667761.1, XP_006900489.1, XP_006215013.1, XP_005408100.1, XP_007628718.1, XP_003514769.1, XP_006160935.1, XP_004683489.1, XP_{—} 003464329.1, XP_004921258.1, XP_003801447.1, XP_006167892.1, XP_004921305.1, AAH89619.1, XP_004706162.1, XP_003583243.1, EFB16804.1, XP_006728603.1, EPQ05185.1, XP_002709040.1, XP_006875861.1, XP_005408103.1, XP_004391425.1, EDL41477.1, XP_006772158.1, EGW06527.1, AAH15294.1, XP_006772162.1, XP_005660939.1, XP_005352050.1, XP_006091901.1, XP_005878675.1, XP_004051323.1, EHA98803.1, XP_003779925.1, EDM12924.1, XP_003421400.1, XP_006160939.1, XP_006160938.1, XP_006160937.1, XP_006160936.1, XP_005702185.1, XP_005313023.1, XP_003769190.1, XP_002714424.1, XP_004715051.1, XP_007661593.1, XP_004590594.1, ELK23975.1, XP_004674085.1, XP_004780477.1, XP_006231186.1, XP_003803573.1, XP_004803176.1, EFB16803.1, XP_006056694.1, XP_005441626.1, XP_005318647.1 XP_004605904.1, XP_005862182.1, XP_003430682.1, XP_004780478.1, XP_005239278.1, XP_003897760.1, XP_007484121.1, XP_004892683.1, XP_004414286.1, XP_006927013.1, XP_003923145.1, XP_852587.2, AAP97178.1, EHH53105.1, XP_005408113.1, XP_002915474.1, XP_005377590.1, XP_527404.2, XP_005552830.1, XP_004044211.1, NP_{—} 001180996.1, XP_003513513.2, XP_001498599.2, XP_002746654.1, XP_005072349.1, XP_006149181.1, EAX04334.1, XP_003833230.1, XP_005216635.1, XP_003404197.1, XP_007523363.1, XP_007433902.1, XP_003254235.1, XP_004471242.1, XP_005216634.1, XP_006860675.1, XP_004771956.1, XP_006038833.1, NP_001138534.1, XP_007068532.1, XP_003510714.1, ERE87950.1, XP_003986313.1, XP_006728644.1, XP_004878099.1, XP_003468014.1, XP_{—} 007095614.1, XP_004648849.1, XP_004869795.1, XP_004018927.1, XP_005696454.1, XP_006201985.1, XP_005960697.1, XP_004813725.1, XP_005496926.1, ELR45088.1, XP_004696625.1, XP_005860982.1, XP_005911003.1, XP_006260162.1, EPQ04414.1, XP_006099775.1, NP_001138532.1, XP_006190795.1, XP_004649775.1, XP_004424497.1, XP_004390885.1, XP_005911004.1, XP_003777803.1, XP_004312259.1, XP_005529140.1, XP_005314582.1, XP_006926523.1, XP_006926522.1, XP_004683491.1, XP_003826680.1, XP_{—} 003215018.1, XP_003215087.1, EGW12611.1, XP_006113023.1, XP_006882182.1, XP_007425200.1, XP_006041342.1, NP_001138533.1, EMP27694.1, XP_007497753.1, XP_006034252.1, or a variant thereof. Throughout this disclosure, any data base code, unless specified to the contrary, refers to a sequence available from the NCBI data bases, more specifically the version online on 5 Aug. 2013, and comprises, if such sequence is a nucleotide sequence, the polypeptide sequence obtained by translating the former.

In particular, the first genetic mutation may increase the activity of glycine-N-acyl transferase (E₂ₐ) and acyl-CoA synthetase (E₃) relative to the wild type cell. More in particular, the sequence of glycine-N-acyl transferase (E₂ₐ) used according to any aspect of the present invention may have 50%, 55%, 60%, 65%, 70%, 75%, 80%, 85%, 90%, 95%, 100% sequence identity with SEQ ID NO:1 and/or the sequence of acyl-CoA synthetase (E₃) used according to any aspect of the present invention may have 50%, 55%, 60%, 65%, 70%, 75%, 80%, 85%, 90%, 95%, 100% sequence identity with SEQ ID NO:2.

In one example, the glycine-N-acyl transferase (E₂ₐ) may be capable of producing N-acyl glutamate. In this example, the glycine-N-acyl transferase (E₂ₐ) may comprise 50%, 55%, 60%, 65%, 70%, 75%, 80%, 85%, 90%, 95%, 100% sequence identity with SEQ ID NO:1.

The cell according to any aspect of the present invention may comprise a genetic mutation in a second amino acid acyl transferase that may be capable of working on other amino acids or variants thereof in conjunction with the activity of the lipase.

The acyl-CoA substrate consumed in the cell according to any aspect of the present invention may be purified from a cell, chemically synthesised or produced using an acyl-CoA synthetase. The term “acyl-CoA synthetase”, as used herein, refers to an enzyme capable of catalysing the ATP- or GTP-dependent conversion of a fatty acid and CoA or ACP to acyl-CoA or acyl-ACP. In one example, the acyl-CoA synthetase may comprise amino acid sequence SEQ ID NO:2.

According to any aspect of the present invention, the term ‘acyl-CoA synthetase’ may refer to an acyl-CoA/ACP synthetase that may be capable of producing acyl thioester, i.e. acyl-CoA or acyl-ACP and/or catalysing the following reaction:

fatty acid+CoA/ACP+ATP/GTP→acyl-CoA/ACP+ADP/GDP+Pi

Examples of acyl-CoA synthetases may include EC 6.2.1.3, EC 6.2.1.10, EC 6.2.1.15, EC 6.2.1.20 and the like. The state of the art describes various methods to detect acyl-CoA synthetase activity. For example, the activity of an acyl-CoA synthetase may be assayed as follows: the standard reaction mixture for the spectrophotometric assay (total volume, 1 ml) is composed of 0.1 M Tris-HCl buffer pH 8.0, 1.6 mM Triton X-100, 5 mM dithiothreitol, 0.15 M KCl, 15 mM MgCl₂, 10 mM ATP, 0.1 mM potassium palmitate, 0.6 mM CoA, 0.2 mM potassium phosphoenolpyruvate, 0.15 mM NADH, 45 pg adenylate kinase per mL, 30 pg pyruvate kinase per mL and 30 pg lactate dehydrogenase per mL. The oxidation of NADH at 334 nm is followed with a recording spectrophotometer.

Alternatively, the activity of an acyl-CoA synthetase may be assayed as described in the state of the art, for example Kang, Y., et al, 2010. Briefly, the amount of free thiol in the form of unreacted CoASH may be determined by adding Ellmann's reagent and spectrophotometrically monitoring the absorbance at 410 nm, in a reaction buffer comprising 150 mM Tris-HCl (pH 7.2), 10 mM MgCl₂, 2 mM EDTA, 0.1% Triton X-100, 5 mM ATP, 0.5 mM Coenzyme A (CoASH) and a fatty acid (30 to 300 mM).

Various acyl-CoA synthetases have been described in the state of the art, for example YP_001724804.1, WP_001563489.1 and NP_707317.1. In one example, the acyl-CoA synthetase comprises SEQ ID NO: 2, YP_001724804.1, BAA15609.1 or a variant thereof.

The cell according to any aspect of the present invention may be genetically different from the wild type cell. The genetic difference between the cell according to any aspect of the present invention and the wild type cell may be in the presence of a complete gene, amino acid, nucleotide etc. in the cell according to any aspect of the present invention that may be absent in the wild type cell. In one example, the cell according to any aspect of the present invention may comprise enzymes that enable the cell to produce at least one acyl amino acid and cell may further comprise at least one mutation that results in the conversion of naturally occurring oils to at least one fatty acid and glycerol. The wild type cell relative to the cell according to any aspect of the present invention may have none or no detectable activity of the enzymes that enable the cell according to any aspect of the present invention to produce at least one acyl amino acid; and the wild type cell may not be capable of hydrolysing oils to fatty acids as per normal.

The phrase “wild type” as used herein in conjunction with a cell may denote a cell with a genome make-up that is in a form as seen naturally in the wild. The term may be applicable for both the whole cell and for individual genes. The term “wild type” therefore does not include such cells or such genes where the gene sequences have been altered at least partially by man using recombinant methods.

A skilled person would be able to use any method known in the art to genetically modify a cell. According to any aspect of the present invention, the cell may be genetically modified so that in a defined time interval, within 2 hours, in particular within 8 hours or 24 hours, it forms at least twice, especially at least 10 times, at least 100 times, at least 1000 times or at least 10000 times more acyl amino acids than the wild-type cell. The increase in product formation can be determined for example by cultivating the cell according to any aspect of the present invention and the wild-type cell each separately under the same conditions (same cell density, same nutrient medium, same culture conditions) for a specified time interval in a suitable nutrient medium and then determining the amount of target product (acyl amino acid) in the nutrient medium.

The phrase ‘decreased activity of an enzyme’ and like may refer to a genetic modification that may be present in the cell according to any aspect of the present invention to decrease a specific enzymatic activity and this may be done by a gene disruption or a genetic modification. In particular, the decrease in activity of an enzyme relative to the wild type cell may be a 10, 15, 20, 25, 30, 35, 40, 45, 50, 55, 60, 65, 70, 75, 80, 85, 90, 95 or 100% less than the wild type cell.

The phrase “increased activity of an enzyme”, as used herein is to be understood as increased intracellular activity. Basically, an increase in enzymatic activity can be achieved by increasing the copy number of the gene sequence or gene sequences that code for the enzyme, using a strong promoter, increasing translation by improved ribosome binding sites or optimized codon usage or employing a gene or allele that code for a corresponding enzyme with increased activity and optionally by combining these measures. Genetically modified cells used according to any aspect of the present invention are for example produced by transformation, transduction, conjugation or a combination of these methods with a vector that contains the desired gene, an allele of this gene or parts thereof and a vector that makes expression of the gene possible. Heterologous expression is in particular achieved by integration of the gene or of the alleles in the chromosome of the cell or an extra-chromosomally replicating vector. In particular, an increase in an activity of an enzyme relative to the wild type cell may be a 10, 15, 20, 25, 30, 35, 40, 45, 50, 55, 60, 65, 70, 75, 80, 85, 90, 95 or 100% more than the wild type cell.

In one example, the cell according to any aspect of the present invention may comprise a second and third genetic mutation that results in the overexpression of at least the enzymes amino acid-N-acyl transferase (E₂) and acyl-CoA synthetase (E₃). In particular, the cell may over express enzymes glycine N-acyl transferase (E₂ₐ) and acyl-CoA/ACP synthetase (E₃). The expression of glycine-N-acyl transferase may be measured using at least the assay disclosed in Badenhorst C P, 2012. Namely, DTNB, water and cell lysate are mixed together and incubated at 37° C. for 10 min while monitoring absorbance at 412 nm.

In particular, the amino acid-N-acyl-transferase (E₂) may be a human glycine N-acyl-transferase (E₂ₐ). In one example, E₁ may comprise SEQ ID NO:4 or a variant thereof. In another example, E₂ may comprise SEQ ID NO:1 and the acyl-CoA synthetase (E₃) may comprise SEQ ID NO: 2 or a variant thereof. More in particular, the cell according to any aspect of the present invention may comprise a first genetic mutation in E₁ which may comprise SEQ ID NO:4 and a second and third genetic mutation in E₁ and E₂, wherein E₁ may comprise a amino acid sequence of SEQ ID NO: 1 and E₂ may comprise amino acid sequence of SEQ ID NO: 2.

According to any aspect of the present invention, a formula referring to a chemical group that represents the dissociated or undissociated state of a compound capable of dissociating in an aqueous solution comprises both the dissociated and the undissociated state and the various salt forms of the group. For example, the residue —COOH comprises both the protonated (—COOH) as well as the unprotonated (—COO⁻) carboxylic acid.

The term “acyl amino acid”, as used herein, refers to the product of the reaction catalysed by an amino acid-N-acyl transferase, a compound represented by the formula acyl-CO—NH—CHR—COOH, wherein R is the side chain of a proteinogenic amino acid, and wherein the term “acyl” refers to the acyl residue of a fatty acid. In one example, the term “fatty acid”, as used herein, means a carboxylic acid, for example an alkanoic acid, with at least 6, 8, 10, or 12 carbon atoms. In one example, it is a linear fatty acid, in another example, it is branched. In one example it is a saturated fatty acid. In another example, it is unsaturated. In one example, it is a fatty acid with 6, 7, 8, 9, 10, 11, 12, 13, 14, 15, 16, 17, 18, 19, 20, 21, 22, 23, 24, 25, 26, 27, 28, 29, 30 carbon atoms. In particular, the acyl amino acid may be N-acyl glycinate, N-acyl glutamate, N-acyl Alaninate, N-acyl Sarcosinate, N-acyl Arginate, N-acyl Lysinate, N-acyl Threoninate, N-acyl Prolinate and the like. In one example, the acyl amino acid produced according to any aspect of the present invention may be selected from the group consisting of cocoyl glycinate, cocoyl glutamate, cocoyl alaninate, cocoyl sarcosinate, cocoyl arginate, cocoyl lysinate, cocoyl threoninate, and cocoyl prolinate.

The teachings of the present invention may not only be carried out using biological macromolecules having the exact amino acid or nucleic acid sequences referred to in this application explicitly, for example by name or accession number, or implicitly, but also using variants of such sequences. The term “variant”, as used herein, comprises amino acid or nucleic acid sequences, respectively, that are at least 70, 75, 80, 85, 90, 92, 94, 95, 96, 97, 98 or 99% identical to the reference amino acid or nucleic acid sequence, wherein preferably amino acids other than those essential for the function, for example the catalytic activity of a protein, or the fold or structure of a molecule are deleted, substituted or replaced by insertions or essential amino acids are replaced in a conservative manner to the effect that the biological activity of the reference sequence or a molecule derived therefrom is preserved. The state of the art comprises algorithms that may be used to align two given nucleic acid or amino acid sequences and to calculate the degree of identity, see Arthur Lesk (2008), Thompson et al., 1994, and Katoh et al., 2005. The term “variant” is used synonymously and interchangeably with the term “homologue”. Such variants may be prepared by introducing deletions, insertions or substitutions in amino acid or nucleic acid sequences as well as fusions comprising such macromolecules or variants thereof. In one example, the term “variant”, with regard to amino acid sequence, comprises, in addition to the above sequence identity, amino acid sequences that comprise one or more conservative amino acid changes with respect to the respective reference or wild type sequence or comprises nucleic acid sequences encoding amino acid sequences that comprise one or more conservative amino acid changes. In one example, the term “variant” of an amino acid sequence or nucleic acid sequence comprises, in addition to the above degree of sequence identity, any active portion and/or fragment of the amino acid sequence or nucleic acid sequence, respectively, or any nucleic acid sequence encoding an active portion and/or fragment of an amino acid sequence. The term “active portion”, as used herein, refers to an amino acid sequence or a nucleic acid sequence, which is less than the full length amino acid sequence or codes for less than the full length amino acid sequence, respectively, wherein the amino acid sequence or the amino acid sequence encoded, respectively retains at least some of its essential biological activity. For example an active portion and/or fragment of a protease may be capable of hydrolysing peptide bonds in polypeptides. The phrase “retains at least some of its essential biological activity”, as used herein, means that the amino acid sequence in question has a biological activity exceeding and distinct from the background activity and the kinetic parameters characterising said activity, more specifically k_{cat} and K_{M}, are preferably within 3, 2, or 1 order of magnitude of the values displayed by the reference molecule with respect to a specific substrate. Similarly, the term “variant” of a nucleic acid comprises nucleic acids the complementary strand of which hybridises, preferably under stringent conditions, to the reference or wild type nucleic acid. A skilled person would be able to easily determine the amino acid-N-acyl-transferases that will be capable of making proteinogenic amino acids and/or fatty acids. In particular, the variants may include but are not limited to an amino acid-N-acyl-transferase selected from the group of organisms consisting of Nomascus leucogenys (NI, XP_003275392.1), Saimiri boliviensis (Sb, XP_003920208.1), Felis catus (Fc, XP_003993512.1), Bos taurus (Bt, NP_001178259.1), Mus musculus (Mm, NP_666047.1) and the like.

Stringency of hybridisation reactions is readily determinable by one ordinary skilled in the art, and generally is an empirical calculation dependent on probe length, washing temperature and salt concentration. In general, longer probes require higher temperatures for proper annealing, while shorter probes need lower temperatures. Hybridisation generally depends on the ability of denatured DNA to reanneal to complementary strands when present in an environment below their melting temperature. The higher the degree of desired homology between the probe and hybridisable sequence, the higher the relative temperature which may be used. As a result it follows that higher relative temperatures would tend to make the reaction conditions more stringent, while lower temperature less so. For additional details and explanation of stringency of hybridisation reactions, see F. M. Ausubel (1995). The person skilled in the art may follow the instructions given in the manual “The DIG System Users Guide for Filter Hybridization”, Boehringer Mannheim GmbH, Mannheim, Germany, 1993 and in Liebl et al., 1991 on how to identify DNA sequences by means of hybridisation. In one example, stringent conditions are applied for any hybridisation, i.e. hybridisation occurs only if the probe is 70% or more identical to the target sequence. Probes having a lower degree of identity with respect to the target sequence may hybridise, but such hybrids are unstable and will be removed in a washing step under stringent conditions, for example by lowering the concentration of salt to 2×SSC or, optionally and subsequently, to 0.5×SSC, while the temperature is, in order of increasing preference, approximately 50° C.-68° C., approximately 52° C.-68° C., approximately 54° C.-68° C., approximately 56° C.-68° C., approximately 58° C.-68° C., approximately 60° C.-68° C., approximately 62° C.-68° C., approximately 64° C.-68° C., approximately 66° C.-68° C. In a particularly preferred embodiment, the temperature is approximately 64° C.-68° C. or approximately 66° C.-68° C. It is possible to adjust the concentration of salt to 0.2×SSC or even 0.1×SSC. Polynucleotide fragments having a degree of identity with respect to the reference or wild type sequence of at least 70, 80, 90, 91, 92, 93, 94, 95, 96, 97, 98, 99% may be isolated. The term “homologue” of a nucleic acid sequence, as used herein, refers to any nucleic acid sequence that encodes the same amino acid sequence as the reference nucleic acid sequence, in line with the degeneracy of the genetic code.

The cell according to any aspect of the present invention may comprise a further genetic mutation that increases the expression of at least one transporter protein relative to the wild type cell. This further mutation enables the cell to increase the uptake of at least one fatty acid. In particular, the transporter protein may be AlkL (SEQ ID NO: 5 or 13) and/or FadL (SEQ ID NO: 6). AlkL and/or FadL may function as at least one transporter protein compared to the wild type cell. In one example, the cell may be genetically modified to overexpress both the fadL and the alkL gene.

The cell according to any aspect of the present invention may have reduced capacity of fatty acid degradation by beta-oxidation relative to the wild type cell. In particular, the reduced fatty acid degradation activity compared to the wild type cell, may be a result of decreased expression relative to the wild type cell of at least one enzyme (E₄) selected from the group consisting of acyl-CoA dehydrogenase (FadE) (E₄ₐ), enoyl-CoA hydratase (FadB) (E_{4b}), (R)-3-hydroxyacyl-CoA dehydrogenase (FadB) (E_{4c}) and 3-ketoacyl-CoA thiolase (FadA) (E_{4d}).

The term “having a reduced fatty acid degradation capacity”, as used herein, means that the respective cell degrades fatty acids, in particular those taken up from the environment, at a lower rate than a comparable cell or wild type cell having normal fatty acid degradation capacity would under identical conditions. In one example, the fatty acid degradation of such a cell is lower on account of deletion, inhibition or inactivation of at least one gene encoding an enzyme involved in the β-oxidation pathway. In one example, at least one enzyme involved in the β-oxidation pathway has lost, in order of increasing preference, 5, 10, 20, 40, 50, 75, 90 or 99% activity relative to the activity of the same enzyme under comparable conditions in the respective wild type microorganism. The person skilled in the art may be familiar with various techniques that may be used to delete a gene encoding an enzyme or reduce the activity of such an enzyme in a cell, for example by exposition of cells to radioactivity followed by accumulation or screening of the resulting mutants, site-directed introduction of point mutations or knock out of a chromosomally integrated gene encoding for an active enzyme, as described in Sambrook/Fritsch/Maniatis (1989). In addition, the transcriptional repressor FadR may be over expressed to the effect that expression of enzymes involved in the β-oxidation pathway is repressed (Fujita, Y., et al, 2007). The phrase “deletion of a gene”, as used herein, means that the nucleic acid sequence encoding said gene is modified such that the expression of active polypeptide encoded by said gene is reduced. For example, the gene may be deleted by removing in-frame a part of the sequence comprising the sequence encoding for the catalytic active centre of the polypeptide. Alternatively, the ribosome binding site may be altered such that the ribosomes no longer translate the corresponding RNA. It would be within the routine skills of the person skilled in the art to measure the activity of enzymes expressed by living cells using standard essays as described in enzymology text books, for example Cornish-Bowden, 1995.

Degradation of fatty acids is accomplished by a sequence of enzymatically catalysed reactions. First of all, fatty acids are taken up and translocated across the cell membrane via a transport/acyl-activation mechanism involving at least one outer membrane protein and one inner membrane-associated protein which has fatty acid-CoA ligase activity, referred to in the case of E. coli as FadL and FadD/FadK, respectively. Inside the cell, the fatty acid to be degraded is subjected to enzymes catalysing other reactions of the β-oxidation pathway. The first intracellular step involves the conversion of acyl-CoA to enoyl-CoA through acyl-CoA dehydrogenase, the latter referred to as FadE in the case of E. coli. The activity of an acyl-CoA dehydrogenase may be assayed as described in the state of art, for example by monitoring the concentration of NADH spectrophotometrically at 340 nm in 100 mM MOPS, pH 7.4, 0.2 mM Enoyl-CoA, 0.4 mM NADH. The resulting enoyl-CoA is converted to 3-ketoacyl-CoA via 3-hydroxyacyl-CoA through hydration and oxidation, catalysed by enoyl-CoA hydratase/(R)-3-hydroxyacyl-CoA dehydrogenase, referred to as FadB and FadJ in E. coli. Enoyl-CoA hydratase/3-hydroxyacyl-CoA dehydrogenase activity, more specifically formation of the product NADH may be assayed spectrophotometrically as described in the state of the art, for example as outlined for FadE. Finally, 3-ketoacyl-CoA thiolase, FadA and FadI in E. coli, catalyses the cleavage of 3-ketoacyl-CoA, to give acetyl-CoA and the input acyl-CoA shortened by two carbon atoms. The activity of ketoacyl-CoA thiolase may be assayed as described in the state of the art, for example in Antonenkov, V., et al, 1997.

The phrase “a cell having a reduced fatty acid degradation capacity”, as used herein, refers to a cell having a reduced capability of taking up and/or degrading fatty acids, particularly those having at least eight carbon chains. The fatty acid degradation capacity of a cell may be reduced in various ways. In particular, the cell according to any aspect of the present invention has, compared to its wild type, a reduced activity of an enzyme involved in the β-oxidation pathway. The term “enzyme involved in the β-oxidation pathway”, as used herein, refers to an enzyme that interacts directly with a fatty acid or a derivative thereof formed as part of the degradation of the fatty acid via the β-oxidation pathway. The β-oxidation pathway comprises a sequence of reactions effecting the conversion of a fatty acid to acetyl-CoA and the CoA ester of the shortened fatty acid. The enzyme involved in the β-oxidation pathway may by recognizing the fatty acid or derivative thereof as a substrate, converts it to a metabolite formed as a part of the β-oxidation pathway. For example, the acyl-CoA dehydrogenase is an enzyme involved in the β-oxidation pathway as it interacts with fatty acid-CoA and converts fatty acid-CoA ester to enoyl-CoA, which is a metabolite formed as part of the β-oxidation. In another example, the term “enzyme involved in the β-oxidation pathway”, as used herein, comprises any polypeptide from the group comprising acyl-CoA dehydrogenase, enoyl-CoA hydratase, 3-hydroxyacyl-CoA dehydrogenase and 3-keto-acyl-CoA thiolase. The acyl-CoA synthetase may catalyse the conversion of a fatty acid to the CoA ester of a fatty acid, i.e. a molecule, wherein the functional group —OH of the carboxy group is replaced with —S-CoA and introducing the fatty acid into the β-oxidation pathway. For example, the polypeptides FadD and FadK in E. coli (accession number: BAA15609.1 and NP_416216.4, respectively) are acyl-CoA synthetases. In one example, the term “acyl-CoA dehydrogenase”, as used herein, may be a polypeptide capable of catalysing the conversion of an acyl-CoA to enoyl-CoA, as part of the β-oxidation pathway. For example, the polypeptide FadE in E. coli (accession number: BAA77891.2) may be an acyl-CoA dehydrogenase. The term “enoyl-CoA hydratase”, as used herein, also referred to as 3-hydroxyacyl-CoA dehydrogenase, refers to a polypeptide capable of catalysing the conversion of enoyl-CoA to 3-ketoacyl-CoA through hydration and oxidation, as part of the β-oxidation pathway. For example, the polypeptides FadB and FadJ in E. coli (accession numbers: BAE77457.1 and P77399.1, respectively) are enoyl-CoA hydratases. The term “ketoacyl-CoA thiolase”, as used herein, may refer to a polypeptide capable of catalysing the cleaving of 3-ketoacyl-CoA, resulting in an acyl-CoA shortened by two carbon atoms and acetyl-CoA, as the final step of the β-oxidation pathway. For example, the polypeptides FadA and FadI in E. coli (accession number: YP_491599.1 and P76503.1, respectively) are ketoacyl-CoA thiolases.

Any of the enzymes used according to any aspect of the present invention, may be an isolated enzyme. In particular, the enzymes used according to any aspect of the present invention may be used in an active state and in the presence of all cofactors, substrates, auxiliary and/or activating polypeptides or factors essential for its activity. The term “isolated”, as used herein, means that the enzyme of interest is enriched compared to the cell in which it occurs naturally. The enzyme may be enriched by SDS polyacrylamide electrophoresis and/or activity assays. For example, the enzyme of interest may constitute more than 5, 10, 20, 50, 75, 80, 85, 90, 95 or 99 percent of all the polypeptides present in the preparation as judged by visual inspection of a polyacrylamide gel following staining with Coomassie blue dye.

The enzyme used according to any aspect of the present invention may be recombinant. The term “recombinant” as used herein, refers to a molecule or is encoded by such a molecule, particularly a polypeptide or nucleic acid that, as such, does not occur naturally but is the result of genetic engineering or refers to a cell that comprises a recombinant molecule. For example, a nucleic acid molecule is recombinant if it comprises a promoter functionally linked to a sequence encoding a catalytically active polypeptide and the promoter has been engineered such that the catalytically active polypeptide is overexpressed relative to the level of the polypeptide in the corresponding wild type cell that comprises the original unaltered nucleic acid molecule.

Whether or not a nucleic acid molecule, polypeptide, more specifically an enzyme used according to any aspect of the present invention, is recombinant or not has not necessarily implications for the level of its expression. However, in one example one or more recombinant nucleic acid molecules, polypeptides or enzymes used according to any aspect of the present invention may be overexpressed. The term “overexpressed”, as used herein, means that the respective polypeptide encoded or expressed is expressed at a level higher or at higher activity than would normally be found in the cell under identical conditions in the absence of genetic modifications carried out to increase the expression, for example in the respective wild type cell. The person skilled in the art is familiar with numerous ways to bring about overexpression. For example, the nucleic acid molecule to be overexpressed or encoding the polypeptide or enzyme to be overexpressed may be placed under the control of a strong inducible promoter such as the lac promoter. The state of the art describes standard plasmids that may be used for this purpose, for example the pET system of vectors exemplified by pET-3a (commercially available from Novagen). Whether or not a nucleic acid or polypeptide is overexpressed may be determined by way of quantitative PCR reaction in the case of a nucleic acid molecule, SDS polyacrylamide electrophoreses, Western blotting or comparative activity assays in the case of a polypeptide. Genetic modifications may be directed to transcriptional, translational, and/or post-translational modifications that result in a change of enzyme activity and/or selectivity under selected and/or identified culture conditions. Thus, in various examples of the present invention, to function more efficiently, a microorganism may comprise one or more gene deletions. Gene deletions may be accomplished by mutational gene deletion approaches, and/or starting with a mutant strain having reduced or no expression of one or more of these enzymes, and/or other methods known to those skilled in the art.

The cell according to any aspect of the present invention may refer to a wide range of microbial cells. In particular, the cell may be selected from the group consisting of Pseudomonas, Corynebacterium, Bacillus and Escherichia. In one example, the cell may be Escherichia coli. In another example, the cell may be a lower eukaryote, such as a fungus from the group comprising Saccharomyces, Candida, Pichia, Schizosaccharomyces and Yarrowia, particularly, Saccharomyces cerevisiae. The microorganism may be an isolated cell, in other words a pure culture of a single strain, or may comprise a mixture of at least two strains. Biotechnologically relevant cells are commercially available, for example from the American Type Culture Collection (ATCC) or the German Collection of Microorganisms and Cell Cultures (DSMZ). Particles for keeping and modifying cells are available from the prior art, for example Sambrook/Fritsch/Maniatis (1989).

In one example, the cell according to any aspect of the present invention may be capable of producing sufficient fatty acids from the natural oil used as a substrate that no further fatty acid has to be added as a substrate. In another example, the amount of fatty acids and/ amino acids used as substrate may be supplemented to increase the production of acyl amino acids.

In this example, the cell may thus be genetically modified to:
increase the expression relative to the wild type cell of an amino acid-N-acyl-transferase (E₂) and an acyl-CoA synthetase (E₃),
increase the expression relative to the wild type cell of a lipase (E₁), and
increase the expression relative to the wild type cell of an enzyme associated to production of proteinogenic amino acids and/or fatty acids from at least one carbohydrate.

The term “proteinogenic amino acid”, as used herein, refers to an amino acid selected from the group comprising alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine and valine. Proteinogenic amino acids and fatty acids are synthesized as part of the primary metabolism of many wild type cells in reactions and using enzymes that have been described in detail in biochemistry textbooks, for example Jeremy M Berg, et al., 2002. In particular, the proteinogenic amino acid may be selected from the group consisting of glycine, glutamine, glutamate, asparagine and alanine. More in particular, the proteinogenic amino acid may be glycine.

The cell according to any aspect of the present invention may comprise at least one genetic mutation that enables the cell to produce at least one fatty acid according to any method known in the art. In one example, the genetic mutation may enable the cell to produce at least one fatty acid by means of a malonyl-CoA dependent and malonyl-ACP independent fatty acyl-CoA metabolic pathway. This is further described in US20140051136.

In another example, the cell according to any aspect of the present invention may be capable of producing its own amino acid. Different enzymes may be selected based on the amino acid to be produced by the cell. In one example, the amino acid to be produced by the cell may be glycine. In particular, the cell according to any aspect of the present invention may comprise a fourth genetic mutation to increase the expression relative to the wild type cell of at least one enzyme (E₆) that may be capable of enabling the cell to produce glycine. More in particular, the enzyme (E₆) may be at least one enzyme from the glycine cleavage system also referred to as the glycine synthase. Even more in particular, the enzyme may be selected from the group consisting of T-protein (EC 2.1.2.10) (E₆ₐ) an aminomethyltransferase, P-protein (EC 1.4.4.2) (E_{6b}) a glycine dehydrogenase, L-protein (EC 1.8.1.4) (Esc) a dihydrolipoyl dehydrogenase and H-protein (E₆ₐ). The cell according to any aspect of the present invention may have an increased expression of any one of the enzymes (E₆ₐ-E₆ₐ) relative to the wild type cell to result in the production of glycine in the cell according to any aspect of the present invention. In another example, the enzyme (E₆) may at least be one serine hydroxymethyltransferase (GlyA) (E₆ₑ) (EC:2.1.2.1) where serine may be used as an intermediate. The cell according to any aspect of the present invention may have an increased expression of E₆ₐ relative to the wild type cell to result in the production of glycine in the cell according to any aspect of the present invention. In another example, the enzyme (E₆) may be a threonine adolase (LtaE) (E_{6f}) (EC: 4.1.2.5) or threonine dehydrogenase (Tdh) (E_{6g}) (EC: 1.1.1.103) and glycine C-acyltransferase (E₆ₕ) also known as 2-Amino-3-Ketobutyrate CoA-Ligase (Kbl) (EC: 2.3.1.29), The cell according to any aspect of the present invention may have an increased expression of E_{6f} or E_{6g} and E₆ₕ relative to the wild type cell to result in the production of glycine in the cell according to any aspect of the present invention. In another example, the cell according to any aspect of the present invention may have an increased expression of E_{6f} and E_{6g} in combination with Allothreonine dehydrogenase (L-allo-threonine dehydrogenase, (YdfG)).

In another example, the forth genetic mutation in the cell according to any aspect of the present invention may comprise a combination of enzymes (E₆ₐ to E₆ₕ) that allow the cell to produce glycine.

The forth mutation in the cell according to any aspect of the present invention may be in any enzyme that results in the cell producing a target amino acid. In one example, the cell may be capable of producing an amino acid and a fatty acid that may provide the substrates for acyl amino acid production.

In another example, the cell according to any aspect of the present invention may comprise a further mutation in at least one enzyme selected from the group consisting of:
(i) An enzyme (E₇) capable of uptake of glutamate; and
(ii) An enzyme (E₈) capable of interconverting acyl-CoAs and acyl-ACPs.

In particular, E₇ may be a glutamate-translocating ABC transporter or permease; and E₈ may be an acyl-CoA:ACP transacylase.

According to another aspect of the present invention, there is provided a method of producing at least one acyl amino acid in an aqueous medium comprising at least one lipase (E₁) and at least one glyceride and amino acid,
wherein the method comprises a step of:
   contacting at least one cell comprising a genetic mutation that increases the expression relative to a wild type cell of an amino acid-N-acyl-transferase (E₂) and an acyl-CoA synthetase (E₃) with the aqueous medium, and
wherein the lipase (E₁) is capable of hydrolysing the glyceride to at least one fatty acid and the cell is capable of converting the fatty acid and amino acid to the acyl amino acid, and
wherein the cell has a reduced fatty acid degradation capacity.

According to yet another aspect of the present invention, there is provided a method of producing at least one acyl amino acid, wherein the method comprises the step of contacting at least one cell according to any aspect of the present invention to an aqueous medium comprising at least one glyceride and amino acid

According to any aspect of the present invention, the lipase (E₁) may be provided externally (i.e. in the aqueous medium) where the cell is grown for the production of acyl amino acids or the lipase (E₁) may be part of the DNA of the cell grown in the aqueous medium to produce acyl amino acids. In whichever scenario the lipase (E₁) is introduced into the method according to any aspect of the present invention, the lipase may be capable of hydrolysing the glyceride in the aqueous medium to produce at least one fatty acid and glycerol.

In one example, where lipase (E₁) is part of the aqueous medium, the concentration of lipase may vary depending on the amount of natural oils present in the medium. The added amount of enzyme, reaction time, or the like must be set to obtain a balance between the obtained oil product yield and efficiency of concentration of the target fatty acid. In one example, the utilised amount of lipase relative to 1 g of triglyceride may be from 10 to 2,000 units, and particularly it may be from 200 to 700 units. Here, 1 unit is the amount of enzyme that releases 1 μmol of fatty acid in 1 minute. The hydrolysis reaction using lipase requires that the reaction be performed in the presence of a sufficient amount of water for expression of the hydrolysis activity of lipase. Relative to the triglyceride, the amount of water present is from 10 to 200 percent by weight, and particularly may be from 50 to 150 percent by weight.

Optimum temperature of the enzyme reaction is known to depend on the enzyme, and reactions are performed within the temperature range. Although lipase reacts within the temperature range thereof, viscosity of the target oil of the lipase reaction increases at low temperature, and the effectiveness of stirring the oil and enzyme-containing water worsens. Thus, the reaction is normally performed at from 30 to 40° C. For example, when Candida cylindracea-derived lipase is used for concentration of polyunsaturated fatty acid, the reaction temperature may be room temperature or as high as 37° C.

The term “contacting”, as used herein, means bringing about direct contact between the amino acid, the fatty acid and/or the cell according to any aspect of the present invention in an aqueous solution. For example, the cell, the amino acid and the fatty acid may not be in different compartments separated by a barrier such as an inorganic membrane. If the amino acid or fatty acid is soluble and may be taken up by the cell or can diffuse across biological membranes, it may simply be added to the cell according to any aspect of the present invention in an aqueous solution. In case it is insufficiently soluble, it may be solved in a suitable organic solvent prior to addition to the aqueous solution. The person skilled in the art is able to prepare aqueous solutions of amino acids or fatty acids having insufficient solubility by adding suitable organic and/or polar solvents. Such solvents may be provided in the form of an organic phase comprising liquid organic solvent. In one example, the organic solvent or phase may be considered liquid when liquid at 25° C. and standard atmospheric pressure. In another example, a fatty acid may be provided in the form of a fatty acid ester such as the respective methyl or ethyl ester. In another example, the compounds and catalysts may be contacted in vitro, i.e. in a more or less enriched or even purified state, or may be contacted in situ, i.e. they are made as part of the metabolism of the cell and subsequently react inside the cell.

The term “an aqueous solution” is used interchangeably with the term ‘aqueous medium” and refers to any solution comprising water, mainly water as solvent that may be used to keep the cell according to any aspect of the present invention, at least temporarily, in a metabolically active and/or viable state and comprises, if such is necessary, any additional substrates. The person skilled in the art is familiar with the preparation of numerous aqueous solutions, usually referred to as media that may be used to keep inventive cells, for example LB medium in the case of E. coli. It is advantageous to use as an aqueous solution a minimal medium, i.e. a medium of reasonably simple composition that comprises only the minimal set of salts and nutrients indispensable for keeping the cell in a metabolically active and/or viable state, by contrast to complex mediums, to avoid dispensable contamination of the products with unwanted side products. For example, M9 medium may be used as a minimal medium.

The method according to any aspect of the present invention may be used to convert both saturated and unsaturated fatty acids from the naturally occurring oils, to acyl amino acids. In case the end product sought-after is to comprise a higher yield of saturated acyl residues than is present, it may be possible to complement the method according to any aspect of the present invention by hydrogenating the acyl residues of the acyl amino acids. The resultant composition would thus comprise a mixture of saturated acyl residues. The hydrogenation may be carried out according to various state of the art processes, for example those described in U.S. Pat. No. 5,734,070. Briefly, the compound to be hydrogenated may be incubated at 100° C. in the presence of hydrogen and a suitable catalyst, for example a nickel catalyst on silicon oxide as a support.

The fatty acids and/or amino acids that are to be converted to acyl amino acids may be added in the form of naturally occurring oils and an amino acid in the aqueous medium according to any aspect of the present invention. In another example, to supplement the substrate of fatty acid (from the naturally occurring oils), the cell that produces the acyl amino acids is capable of producing the fatty acids from which the acyl amino acids are produced. In particular, the cells may be genetically modified to be able to produce fatty acids and/or amino acids. In one example, the genetic modification may be to decrease a specific enzymatic activity and this may be done by a gene disruption or a genetic modification. The genetic modification may also increase a specific enzymatic activity. In particular, the genetic modification may increase microbial synthesis of a selected fatty acid or fatty acid derived chemical product above a rate of a control or wild type cell. This control or wild type cell may lack this genetic modification to produce a selected chemical product.

According to a further aspect of the present invention, there is provided a use of at least one cell according to any aspect of the present invention for the production of at least one acyl amino acid.

In particular, the acyl amino acid may be fatty acid-acyl glycinate. More in particular, the fatty acid acyl glycinate may be selected from the group consisting of lauroyl glycinate, myristoyl glycinate and palmitoyl glycinate

In particular, the method according to any aspect of the present invention is carried out within the cell according to any aspect of the present invention.

### BRIEF DESCRIPTION OF THE FIGURES

No Figures

### EXAMPLES

The foregoing describes preferred embodiments, which, as will be understood by those skilled in the art, may be subject to variations or modifications in design, construction or operation without departing from the scope of the claims. These variations, for instance, are intended to be covered by the scope of the claims.

### Example 1

Production of Acyl Amino Acids from Coconut Oil

The strain E. coli W3110 ΔfadE pCDF{Ptac}[hGLYAT2(co_Ec)/fadD_Ec] {Plavuv5} [alkLmod1] was fermented in a fed-batch fermentation to study the ability of linking fatty acids from coconut oil and glycine to fatty glycinates using an external lipase from Thermomyces lanuginosus.

The strain was used for studying their ability to produce fatty acid amino acid adducts with glycine and coconut oil. For this purpose, the strain was cultured both in shake flask and in fed-batch fermentation. The fermentation was carried out in a parallel fermentation system from DASGIP with 8 bioreactors.

The fermentation was performed using 1 l reactors equipped with overhead stirrers and impeller blades. pH and pO₂ were measured online for process monitoring. OTR/CTR measurements served for estimating the metabolic activity and cell fitness, inter alia. The pH electrodes were calibrated by means of a two-point calibration using standard solutions of pH 4.0 and pH 7.0, as specified in DASGIP's technical instructions. The reactors were provided with the necessary sensors and connections as specified in the technical instructions, and the agitator shaft was fitted. The reactors were then charged with 300 mL water and autoclaved for 20 min at 121° C. to ensure sterility. The pO₂ electrodes were connected to the measuring amplifiers and polarized overnight (for at least 6 h). Thereafter, the water was removed under a clean bench and replaced by M9 medium (pH 7.4) composed of KH₂PO₄ 3.0 g/l, Na₂HPO₄ 6.79 g/l, NaCl 0.5 g/l, NH₄Cl 2.0 g/l, 2 mL of a sterile 1 M MgSO₄*7H₂O solution and 1 mL/l of a filter-sterilized trace element stock solution (composed of HCl (37%) 36.50 g/L, MnCl₂*4H₂O 1.91 g/L, ZnSO₄*7H₂O 1.87 g/L, ethylenediaminetetraacetic acid dihydrate 0.84 g/L, H₃BO₃ 0.30 g/L, Na₂MoO₄*2H₂O 0.25 g/L, CaCl₂*2H₂O 4.70 g/L, FeSO₄*7H₂O 17.80 g/L, CuCl₂*2H₂O 0.15 g/L) with 15 g/L glucose as the carbon source (added by metering in 30 mL/L of a sterile feed solution composed of 500 g/L glucose, 1.3% (w/v) MgSO₄*7H₂O) supplemented with 100 mg/L spectinomycin and 0.5 mL/L Delamex.

Thereafter, the pO₂ electrodes were calibrated to 100% with a one-point calibration (stirrer: 400 rpm/aeration: 10 sl/h air), and the feed, correction agent and induction agent lines were cleaned by “cleaning in place” as specified in the technical instructions. To this end, the tubes were rinsed first with 70% ethanol, then with 1 M NaOH, then with sterile fully-demineralized water and, finally, filled with the respective media.

Using the E. coli strain W3110 ΔfadE pCDF{Ptac}[hGLYAT2(co_Ec)/fadD_Ec] {Plavuv5} [alkLmod1], LB medium (10 mL in a 100-mL baffle flask) supplemented with 100 mg/l spectinomycin was inoculated 100 μL of a cryoculture and the culture was grown at 37° C. and 200 rpm for approximately 14 h. Thereafter, this culture was used for a second preculture stage with an initial OD of 0.2 in 50 mL of M9 medium, composed of KH₂PO₄ 3.0 g/l, Na₂HPO₄ 6.79 g/l, NaCl 0.5 g/l, NH₄Cl 2.0 g/l, 2 mL of a sterile 1 M MgSO₄*7H₂O solution and 1 mL/l of a filter-sterilized trace element stock solution (composed of HCl (37%) 36.50 g/L, MnCl₂*4H₂O 1.91 g/L, ZnSO₄*7H₂O 1.87 g/L, ethylenediaminetetraacetic acid dihydrate 0.84 g/l, H₃BO₃ 0.30 g/l, Na₂MoO₄*2H₂O 0.25 g/L, CaCl₂*2H₂O 4.70 g/L, FeSO₄*7H₂O 17.80 g/L, CuCl₂*2H₂O 0.15 g/L) supplemented with 15 g/L glucose as carbon source (added by metering in 30 mL/L of a sterile feed solution composed of 500 g/L glucose) together with the above-described antibiotics was transferred into a 500-mL baffle flask and incubated for 8-12 h at 37° C./200 rpm.

To inoculate the reactors with an optical density of 0.1, the OD₆₀₀ of the second preculture stage was measured and the amount of culture required for the inoculation was calculated. The required amount of culture was placed into the heated and aerated reactor with the aid of a 5-mL syringe through a septum.

The standard program shown in Table 1a-c was used:

TABLE 1
Standard program for use of heated and aerated reactor
a)

| | |
|---|---|
| DO controller | pH controller |

| Preset | 0% | Preset | 0 | mL/h |
|---|---|---|---|---|
| P | 0.1 | P | 5 | |
| Ti | 300 s | Ti | 200 | s |
| Min | 0% | Min | 0 | mL/h |
| Max | 100% | Max | 40 | mL/h |

b)

| N | | | XO2 | | | F | | |
|---|---|---|---|---|---|---|---|---|
| (Rotation) | From | To | (gas mixture) | from | to | (gas flow) | from | to |
| Growth and | 0% | 30% | Growth and | 0% | 100% | Growth and | 15% | 80% |
| biotrans- | 400 rpm | 1500 rpm | biotrans- | 21% | 21% | biotrans- | 6 sl/h | 72 sl/h |
| formation | | | formation | | | formation | | |

c)

| Script | | |
|---|---|---|
| Trigger fires | 31% DO (1/60 h) | |
| Temperature | 37° | C. |
| Induction IPTG | 2 h after the feed start | |
| Feed trigger | 50% | DO |
| Feed rate | 3 | [mL/h] |

The pH was adjusted unilaterally to pH 7.0 with 12.5% strength ammonia solution. During the growth phase and the biotransformation, the dissolved oxygen (pO₂ or DO) in the culture was adjusted to at least 30% via the stirrer speed and the aeration rate. After the inoculation, the DO dropped from 100% to these 30%, where it was maintained stably for the remainder of the fermentation.

The fermentation was carried out as a fed batch, the feed start as the beginning of the feed phase with 5 g/l*h glucose feed, composed of 500 g/l glucose, 1.3% (w/v) MgSO₄*7H₂O, being triggered via the DO peak which indicates the end of the batch phase. From the feed start onwards, the temperature was reduced from 37° C. to 30° C. 2 h after the feed start, the expression was induced with 1 mM IPTG.

In the experimental setting the biotransformation starts 2 h after induction 100 g/l glycine in demineralized water (100 mL/L) and 5 g coconut oil (heated) were fed rather than glucose. Start of the reaction was the addition of the coconut oil. At this time 20 μL of a lipase solution (approx. 2000 U) was also added to the medium.

To quantify lauroyl, myristoyl and palmitoyl glycinate in fermentation samples, samples were taken 23 h and 42 h after the start of the fermentation. These samples were analysed and the results are shown in Table 2.

It has been possible to demonstrate that the strain E. coli W3110 ΔfadE pCDF{Ptac}[hGLYAT2(co_Ec)_fadD_Ec] {Plavuv5} [alkLmod1] using an external lipase is capable of linking fatty acids after cleaving of the coconut oil and glycine producing fatty acid glycinates.

TABLE 2
Quantification of fatty acid glycinates after 25, 29, 40 and 45 h fermentation time.

| Fermentation time | C_{Lauroyl glycinate} | C_{Myristoyl glycinate} | C_{Palmitoyl glycinate} | C_{Glycine} | C_{Octanoic acid} | C_{Decanoic acid} | C_{Lauric acid} |
|---|---|---|---|---|---|---|---|
| [h] | [g/L] | [g/L] | [g/L] | [g/L] | [g/L] | [g/L] | [g/L] |
| 25 | 1.79 | 0.86 | 0.41 | 7.6 | <0.1 | <0.1 | <0.1 |
| 29 | 2.16 | 0.92 | 0.47 | 4.7 | <0.1 | <0.1 | <0.1 |
| 40 | 2.62 | 0.85 | 0.43 | <0.1 | <0.1 | <0.1 | <0.1 |
| 45 | 1.96 | 0.62 | 0.32 | n.d. | <0.1 | <0.1 | <0.1 |

| Fermentation time | C_{Myristic acid} | C_{Palmitoleic acid} | C_{Palmitic acid} | C_{Oleic acid} | C_{Stearic acid} |
|---|---|---|---|---|---|
| [h] | [g/L] | [g/L] | [g/L] | [g/L] | [g/L] |
| 25 | <0.1 | <0.1 | <0.1 | <0.1 | <0.1 |
| 29 | <0.1 | <0.1 | <0.1 | <0.1 | <0.1 |
| 40 | <0.1 | <0.1 | <0.1 | <0.1 | <0.1 |
| 45 | <0.1 | <0.1 | <0.1 | <0.1 | <0.1 |

### Example 2

Generation of a Vector for Expression of the Lipase Gene of Thermomyces lanuginosus in Escherichia coli W3110 ΔfadE

To generate a vector for the expression of the Lipase gene of Thermomyces lanuginosus (lipTI) (SEQ ID NO: 4) a synthetic gene-fusion of the target gene lipTI with an E. coli secretion signal ompA was used (Sletta et al. 2007, Movva et al. 1980). The synthetic, E. coli codon-optimized fusion was amplified using the oligonucleotides ompA_fw (SEQ ID NO: 7) and lipTI_rev (SEQ ID NO: 8) to generate overlaps to the target vector pQE80L-kan (SEQ ID NO: 9) for cloning purposes. A PCR fragment of the expected size could be amplified (985 bp, (SEQ ID NO: 10), was separated via agarose gel electrophoresis and isolated with QiaQuick Gel extraction Kit (Qiagen, Hilden). The purified PCR fragment was assembled with the EcoRI/BamHI cut vector pQE80L-kan using the Gibson Assembly®-Kit ((New England Biolabs, Frankfurt) and transformed into chemically competent E. coli DH5α cells (New England Biolabs, Frankfurt). Procedure of PCR purification, in-vitro cloning and transformation were carried out according to manufacturer's manual. The correct insertion of the target genes was checked by restriction analysis and the authenticity of the introduced genes was verified by DNA sequencing. The resulting vector was named pQE80L-kan{PT5}[ompA-lipTI(co_Ec)] (SEQ ID NO: 11).

The plasmids pCDF{Ptac}[hGLYAT2(co_Ec)/fadD_Ec] {Plavuv5} [alkLmod1] and pQE80L-kan{PT5}[ompA-lipTI(co_Ec)] were co-transformed into the strain E. coli W3110 ΔfadE by means of electroporation and plated onto LB-agar plates supplemented with spectinomycin (50-100 μg/mL) and kanamycin (50 μg/mL). Transformants were checked for the presence of the correct plasmids by plasmid preparation and analytic restriction analysis. The resulting strain was named E. coli W3110 ΔfadE pCDF{Ptac}[hGLYAT2(co_Ec)/fadD_Ec] {Plavuv5} [alkLmod1] and pQE80L-kan{PT5}[ompA-LipTI(co_Ec)]. The sequence of the fusion-protein ompA-LipTI is listed as SEQ ID NO: 12.

### REFERENCES

1. Antonenkov, V., et al. (1997) J. Biol. Chem. 1997, 272:26023-26031
2. Arthur Lesk (2008), Introduction to bioinformatics, 3^{rd} edition,
3. F. M. Ausubel (1995), Current Protocols in Molecular Biology. John Wiley & Sons, Inc.
4. Barker H A, (1959) J Biol Chem. February; 234(2):320-8.
5. Barker H A (1967) Arch. Mikrobiol. 59, 4-12
6. Blair A H, Barker H A (1966) J. Biol. Chem. 241, 400-408
7. Badenhorst C P, Drug Metab Dispos. 2012 February; 40(2):346-52.
8. Buckel W, Dorn U and Semmler R (1981) Eur. J. Biochem. 118, 315-321
9. Chen H P and Marsh E N G (1997) Biochemistry 36, 14939-14945
10. Cornish-Bowden (1995), Fundamentals of Enzyme Kinetics, Portland Press Limited, 1995
11. Duff S M, (2012) Arch. Biochem. Biophys. 528, 90-101
12. Esser D, (2013) Extremophiles, 17:205-216
13. Feng Y, (2002) Biochemistry 41. October 22; 41(42):12883-90
14. Fujita, Y., Matsuoka, H., and Hirooka, K. (2007) Mol. Microbiology 66(4), 829-839
15. Georg Thieme Verlag, Allgemeine Mikrobiologie, 2008,
16. Härtel U, (1993) Archives of Microbiology. Volume 159, Issue 2, pp 174-181
17. Hawkins A B, (2014) Appl Environ Microbiol. April; 80(8):2536-45. doi: 10.1128/AEM.04146-13. Epub 2014 Feb. 14
18. Jeffery D, (1988) Insect Biochemistry. Volume 18, Issue 4, 1988, Pages 347-349
19. Jeremy M Berg, John L Tymoczko, and Lubert Stryer, Biochemistry, 5^{th} edition, W. H. Freeman, 2002.
20. Kalliri E, (2008) J. Bacteriol. 190, 3793-3798
21. Kang, Y., (2010), PLOS ONE 5 (10), e13557
22. Karmen (1955) J Clin Invest. January; 34(1):131-3
23. Katoh et al., (2005) Genome Information, 16(1), 22-33,
24. Liebl W., (1991) International Journal of Systematic Bacteriology 41: 255-260
25. Liu W, (2005) Biochemistry. March 1; 44(8):2982-92
26. Mavrides C (1975) J. Biol. Chem. 250, 4128-4133
27. Moskowitz G J (1969) Biochemistry. 8(7):2748-55
28. Movva N R, Nakamura K, Inouye M. (1980). J Biol Chem. January 10; 255(1):27-9.
29. Parthasarathy A, (2011) Biochemistry. May 3; 50(17):3540-50. Epub 2011 Apr. 5
30. Poulsen, K. R., (2005). Biochemistry, 44, 11574-11580
31. Sambrook/Fritsch/Maniatis (1989): Molecular cloning—A Laboratory Manual, Cold Spring Harbour Press, 2^{nd} edition, Fuchs/Schlegel (2007),
32. Sletta H et al (2007). Appl Environ Microbiol. February; 73(3):906-12 Epub 2006 Dec. 1.
33. Söhling B (1993) Eur J Biochem. February 15; 212(1):121-7
34. Taylor R C, (2010) Microbiology 156, 1975-1982
35. Thompson et al., Nucleic Acids Research 22, 4637-4680, 1994,
36. Waluk, D. (2010), FASEB J. 24, 2795-2803.
37. Wang C C, (1969) J. Biol. Chem. 244, 2516-2526
38. Wiesenborn D P, (1988) Appl. Environ. Microbiol. 54, 2717-2722
39. Yamashita H, (2006) Biochim. Biophys. Acta 1761, 17-23
40. Yan et al., (2014), Biotechnology for Biofuels, 7:55
41. Yu K, (2011) Enzyme Microb Technol. August 10; 49(3):272-6.
42. U.S. Pat. No. 5,734,070, US20140051136

1-14. (canceled)

15. A microbial cell for producing at least one acyl amino acid, wherein the microbial cell is genetically modified to comprise:
a) a first genetic mutation that increases expression relative to a wild type cell of at least one lipase (EC 3.1.1) (E₁) capable of hydrolysing at least one glyceride to at least one fatty acid, wherein the glyceride is a triglyceride; and
b) a second and a third genetic mutation that increases expression relative to a wild type cell of:
ii) an amino acid-N-acyl-transferase (EC 2.3.1) (E₂), and
(ii) an acyl-CoA synthetase (EC 6.2.1.3) (E₃),
thereby enabling the cell to convert the fatty acid to at least one acyl amino acid;
and wherein the microbial cell has a reduced fatty acid degradation capacity.

16. The microbial cell of claim 15, wherein the lipase (E₁) is a lipase derived from any one of the microorganisms selected from the group consisting of: Candida cylindracea, Burkholderia cepacia, Pseudomonas fluorescens, Thermomyces lanuginosus, and Rhizomucor miehei.

17. The microbial cell of claim 15, wherein:
a) E₁ comprises SEQ ID NO:4 or a variant thereof, wherein the variant comprises an amino acid sequence at least 70% identical to SEQ ID NO:4;
b) E₂ comprises SEQ ID NO:1 or a variant thereof, wherein the variant comprises an amino acid sequence at least 70% identical to SEQ ID NO:1; and/or
c) E₃ comprises SEQ ID NO:2 or a variant thereof, wherein the variant comprises an amino acid sequence at least 70% identical to SEQ ID NO:2.

18. The microbial cell of claim 15, wherein the microbial cell's fatty acid degradation capacity is reduced owing to a decrease in activity, compared to the wild type cell, of at least one enzyme (E₄) selected from the group consisting of acyl-CoA dehydrogenase (E₄ₐ), 2,4-dienoyl-CoA reductase (E_{4b}), enoyl-CoA hydratase (E_{4c}) and 3-ketoacyl-CoA thiolase (E_{4d}).

19. The microbial cell of claim 15, wherein the amino acid-N-acyl-transferase (E₂) is glycine-N-acyl-transferase.

20. The microbial cell of claim 15, wherein the cell is capable of making proteinogenic amino acids and/or fatty acids.

21. The microbial cell of claim 15, wherein the acyl amino acid is a N-acyl glycinate or N-acyl glutamate.

22. The microbial cell of claim 15, wherein the cell has a further genetic mutation that increases the expression of at least one transporter protein relative to the wild type cell, and said transporter protein enables the microbial cell to increase the uptake of at least one fatty acid.

23. The microbial cell of claim 22, wherein the transporter protein is selected from the group consisting of FadL and AlkL.

24. The microbial cell of claim 16, wherein:
a) E₁ comprises SEQ ID NO:4 or a variant thereof, wherein the variant comprises an amino acid sequence at least 70% identical to SEQ ID NO:4;
b) E₂ comprises SEQ ID NO:1 or a variant thereof, wherein the variant comprises an amino acid sequence at least 70% identical to SEQ ID NO:1; and/or
c) E₃ comprises SEQ ID NO:2 or a variant thereof, wherein the variant comprises an amino acid sequence at least 70% identical to SEQ ID NO:2.

25. The microbial cell of claim 24, wherein the amino acid-N-acyl-transferase (E₂) is glycine-N-acyl-transferase.

26. The microbial cell of claim 25, wherein the acyl amino acid is a N-acyl glycinate or N-acyl glutamate.

27. The microbial cell of claim 26, wherein the cell has a further genetic mutation that increases the expression of at least one transporter protein relative to the wild type cell, and said transporter protein enables the cell to increase the uptake of at least one fatty acid.

28. A method of producing at least one acyl amino acid in an aqueous medium comprising at least one lipase (E₁) and at least one glyceride and/or amino acid, wherein: a) the method comprises contacting at least one cell comprising a genetic mutation that increases expression relative to a wild type cell of an amino acid-N-acyl-transferase (E₂) and an acyl-CoA synthetase (E₃) with the aqueous medium; b) the lipase (E₁) is capable of hydrolysing the glyceride to at least one fatty acid; c) the cell is capable of converting the fatty acid and amino acid to the acyl amino acid; d) the glyceride is a triglyceride; and e) the cell has a reduced fatty acid degradation capacity.

29. The method of claim 28, wherein the lipase (E₁) is a lipase derived from any one of the microorganisms selected from the group consisting of Candida cylindracea, Burkholderia cepacia, Pseudomonas fluorescens, Thermomyces lanuginosus, and Rhizomucor miehei.

30. The method of claim 28, wherein:
a) E₁ comprises SEQ ID NO:4 or a variant thereof, wherein the variant comprises an amino acid sequence at least 70% identical to SEQ ID NO:4;
b) E₂ comprises SEQ ID NO:1 or a variant thereof, wherein the variant comprises an amino acid sequence at least 70% identical to SEQ ID NO:1;
c) E₃ comprises SEQ ID NO:2 or a variant thereof, wherein the variant comprises an amino acid sequence at least 70% identical to SEQ ID NO:2.

31. The method of claim 30, wherein the lipase (E₁) is a lipase derived from any one of the microorganisms selected from the group consisting of: Candida cylindracea, Burkholderia cepacia, Pseudomonas fluorescens, Thermomyces lanuginosus, and Rhizomucor miehei.

32. A method of producing at least one acyl amino acid, wherein the method comprises contacting the microbial cell of claim 15 with an aqueous medium comprising at least one glyceride and an amino acid, wherein the glyceride is a triglyceride.

33. The method of claim 32, wherein the glyceride is a natural oil.

34. The method of claim 33, wherein the lipase (E₁) is a lipase derived from any one of the microorganisms selected from the group consisting of Candida cylindracea, Burkholderia cepacia, Pseudomonas fluorescens, Thermomyces lanuginosus, and Rhizomucor miehei.


SEQUENCE LISTING (13 sequences)

SEQ ID NO: 1 - protein, 294 aa, Homo sapiens
MLVLHNSQKLQILYKSLEKSIPESIKVYGAIFNIKDKNPFNMEVLVDAWPDYQIVITRPQKQEMKDDQDHYTNTYHIFTKAPDKLEEVLSYSNVISWEQTLQIQGCQEGLDEAIRKVATSKSVQVDYMKTILFIPELPKKHKTSSNDKMELFEVDDDNKEGNFSNMFLDASHAGLVNEHWAFGKNERSLKYIERCLQDFLGFGVLGPEGQLVSWIVMEQSCELRMGYTVPKYRHQGNMLQIGYHLEKYLSQKEIPFYFHVADNNEKSLQALNNLGFKICPCGWHQWKCTPKKYC

SEQ ID NO: 2 - protein, 561 aa, Artificial Sequence
  FadD Acyl-CoA synthase
MKKVWLNRYPADVPTEINPDRYQSLVDMFEQSVARYADQPAFVNMGEVMTFRKLEERSRAFAAYLQQGLGLKKGDRVALMMPNLLQYPVALFGILRAGMIVVNVNPLYTPRELEHQLNDSGASAIVIVSNFAHTLEKVVDKTAVQHVILTRMGDQLSTAKGTVVNFVVKYIKRLVPKYHLPDAISFRSALHNGYRMQYVKPELVPEDLAFLQYTGGTTGVAKGAMLTHRNMLANLEQVNATYGPLLHPGKELVVTALPLYHIFALTINCLLFIELGGQNLLITNPRDIPGLVKELAKYPFTAITGVNTLFNALLNNKEFQQLDFSSLHLSAGGGMPVQQVVAERWVKLTGQYLLEGYGLTECAPLVSVNPYDIDYHSGSIGLPVPSTEAKLVDDDDNEVPPGQPGELCVKGPQVMLGYWQRPDATDEIIKNGWLHTGDIAVMDEEGFLRIVDRKKDMILVSGFNVYPNEIEDVVMQHPGVQEVAAVGVPSGSSGEAVKIFVVKKDPSLTEESLVTFCRRQLTGYKVPKLVEFRDELPKSNVGKILRRELRDEARGKVDNKA

SEQ ID NO: 3 - DNA, 906 bp, Artificial Sequence
  synUcTE (an acyl-CoA thioesterase) gene (codon-optimized)
atgactctagagtggaaaccgaaaccaaaactgcctcaactgctggatgatcacttcggtctgcacggtctggtgtttcgtcgtactttcgcaattcgttcttatgaagtgggtccagatcgttctacctccatcctggccgtcatgaaccacatgcaggaagccaccctgaatcacgcgaaatctgttggtatcctgggtgatggtttcggcactactctggaaatgtctaaacgtgacctgatgtgggtagtgcgtcgcacccacgtagcagtagagcgctaccctacttggggtgacactgtggaagtcgagtgttggattggcgcgtccggtaacaatggtatgcgtcgcgattttctggtccgtgactgtaaaacgggcgaaatcctgacgcgttgcacctccctgagcgttctgatgaacacccgcactcgtcgcctgtctaccatcccggacgaagtgcgcggtgagatcggtcctgctttcatcgataacgtggcagttaaagacgacgaaatcaagaaactgcaaaaactgaacgactccaccgcggactacatccagggcggtctgactccgcgctggaacgacctggatgttaatcagcatgtgaacaacctgaaatacgttgcttgggtcttcgagactgtgccggacagcattttcgaaagccatcacatttcctcttttactctggagtaccgtcgcgaatgtactcgcgactccgttctgcgcagcctgaccaccgtaagcggcggttctagcgaggcaggtctggtctgcgaccatctgctgcaactggaaggcggctccgaagtcctgcgtgcgcgtacggagtggcgtccaaagctgacggattctttccgcggcatctccgtaattccggcggaacctcgtgtttaa

SEQ ID NO: 4 - protein, 290 aa, Thermomyces lanuginosus
MRSSLVLFFVSAWTALASPIRREVSQDLFNQFNLFAQYSAAAYCGKNNDAPAGTNITCTGNACPEVEKADATFLYSFEDSGVGDVTGFLALDNTNKLIVLSFRGSRSIENWIGNLNFDLKEINDICSGCRGHDGFTSSWRSVADTLRQKVEDAVREHPDYRVVFTGHSLGGALATVAGADLRGNGYDIDVFSYGAPRVGNRAFAEFLTVQTGGTLYRITHTNDIVPRLPPREFGYSHSSPEYWIKSGTLVVTRNDIVKIEGIDATGGNNQPNIPDIPAHLWYFGLIGTCL

SEQ ID NO: 5 - protein, 230 aa, Pseudomonas putida
MSFSNYKVIAMPVLVANFVLGAATAWANENYPAKSAGYNQGDWVASFNFSKVYVGEELGDLNVGGGALPNADVSIGNDTTLTFDIAYFVSSNIAVDFFVGVPARAKFQGEKSISSLGRVSEVDYGPAILSLQYHYDSFERLYPYVGVGVGRVLFFDKTDGALSSFDIKDKWAPAFQVGLRYDLGNSWMLNSDVRYIPFKTDVTGTLGPVPVSTKIEVDPFILSLGASYVF

SEQ ID NO: 6 - protein, 448 aa, Artificial Sequence
  FadL
MVMSQKTLFTKSALAVAVALISTQAWSAGFQLNEFSSSGLGRAYSGEGAIADDAGNVSRNPALITMFDRPTFSAGAVYIDPDVNISGTSPSGRSLKADNIAPTAWVPNMHFVAPINDQFGWGASITSNYGLATEFNDTYAGGSVGGTTDLETMNLNLSGAYRLNNAWSFGLGFNAVYARAKIERFAGDLGQLVAGQIMQSPAGQTQQGQALAATANGIDSNTKIAHLNGNQWGFGWNAGILYELDKNNRYALTYRSEVKIDFKGNYSSDLNRAFNNYGLPIPTATGGATQSGYLTLNLPEMWEVSGYNRVDPQWAIHYSLAYTSWSQFQQLKATSTSGDTLFQKHEGFKDAYRIALGTTYYYDDNWTFRTGIAFDDSPVPAQNRSISIPDQDRFWLSAGTTYAFNKDASVDVGVSYMHGQSVKINEGPYQFESEGKAWLFGTNFNYAF

SEQ ID NO: 7 - DNA, 45 bp, Artificial Sequence
  ompAfwd
cggataacaatttcacacagaggagaaattaactatgaaaaaaac

SEQ ID NO: 8 - DNA, 40 bp, Artificial Sequence
  LipT1-rev
gggtaccgagctcgcatgcgtcacaggcaggtgccaatca

SEQ ID NO: 9 - DNA, 4541 bp, Artificial Sequence
  vector pQE80L-kan
ctcgagaaatcataaaaaatttatttgctttgtgagcggataacaattataatagattcaattgtgagcggataacaatttcacacagaattcattaaagaggagaaattaactatgagaggatcgcatcaccatcaccatcacggatccgcatgcgagctcggtaccccgggtcgacctgcagccaagcttaattagctgagcttggactcctgttgatagatccagtaatgacctcagaactccatctggatttgttcagaacgctcggttgccgccgggcgttttttattggtgagaatccaagctagcttggcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaatttcgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggggtaatgactctctagcttgaggcatcaaataaaacgaaaggctcagtcgaaagactgggcctttcgttttatctgttgtttgtcggtgaacgctctcctgagtaggacaaatccgccctctagattacgtgcagtcgatgataagctgtcaaacatgagaattgtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcaccattcgatggtgtcggaatttcgggcagcgttgggtcctggccacgggtgcgcatgatctagagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatggcgttaagggattttggtcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccatgacattaacctataaaaataggcgtatcacgaggccctttcgtcttcac

SEQ ID NO: 10 - DNA, 999 bp, Artificial Sequence
  amplified PCR product
cggataacaatttcacacagaggagaaattaactatgaaaaaaactgctatcgctatcgctgttgctctggctggtttcgctactgttgctcaggccgaattcatgcgcagcagcctggtgctgttttttgtgagcgcgtggaccgcgctggcgagcccgattcgccgcgaagtgagccaggatctgtttaaccagtttaacctgtttgcgcagtatagcgcggcggcgtattgcggcaaaaacaacgatgcgccggcgggcaccaacattacctgcaccggcaacgcgtgcccggaagtggaaaaagcggatgcgacctttctgtatagctttgaagatagcggcgtgggcgatgtgaccggctttctggcgctggataacaccaacaaactgattgtgctgagctttcgcggcagccgcagcattgaaaactggattggcaacctgaactttgatctgaaagaaattaacgatatttgcagcggctgccgcggccatgatggctttaccagcagctggcgcagcgtggcggataccctgcgccagaaagtggaagatgcggtgcgcgaacatccggattatcgcgtggtgtttaccggccatagcctgggcggcgcgctggcgaccgtggcgggcgcggatctgcgcggcaacggctatgatattgatgtgtttagctatggcgcgccgcgcgtgggcaaccgcgcgtttgcggaatttctgaccgtgcagaccggcggcaccctgtatcgcattacccataccaacgatattgtgccgcgcctgccgccgcgcgaatttggctatagccatagcagcccggaatattggattaaaagcggcaccctggtgccggtgacccgcaacgatattgtgaaaattgaaggcattgatgcgaccggcggcaacaaccagccgaacattccggatattccggcgcatctgtggtattttggcctgattggcacctgcctgtgacgcatgcgagctcggtaccc

SEQ ID NO: 11 - DNA, 5439 bp, Artificial Sequence
  pQE80L-kan{PT5}[ompA-lipTl(co_Ec)]
cgggtcgacctgcagccaagcttaattagctgagcttggactcctgttgatagatccagtaatgacctcagaactccatctggatttgttcagaacgctcggttgccgccgggcgttttttattggtgagaatccaagctagcttggcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaatttcgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggggtaatgactctctagcttgaggcatcaaataaaacgaaaggctcagtcgaaagactgggcctttcgttttatctgttgtttgtcggtgaacgctctcctgagtaggacaaatccgccctctagattacgtgcagtcgatgataagctgtcaaacatgagaattgtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcaccattcgatggtgtcggaatttcgggcagcgttgggtcctggccacgggtgcgcatgatctagagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatggcgttaagggattttggtcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccatgacattaacctataaaaataggcgtatcacgaggccctttcgtcttcacctcgagaaatcataaaaaatttatttgctttgtgagcggataacaattataatagattcaattgtgagcggataacaatttcacacagaggagaaattaactatgaaaaaaactgctatcgctatcgctgttgctctggctggtttcgctactgttgctcaggccgaattcatgcgcagcagcctggtgctgttttttgtgagcgcgtggaccgcgctggcgagcccgattcgccgcgaagtgagccaggatctgtttaaccagtttaacctgtttgcgcagtatagcgcggcggcgtattgcggcaaaaacaacgatgcgccggcgggcaccaacattacctgcaccggcaacgcgtgcccggaagtggaaaaagcggatgcgacctttctgtatagctttgaagatagcggcgtgggcgatgtgaccggctttctggcgctggataacaccaacaaactgattgtgctgagctttcgcggcagccgcagcattgaaaactggattggcaacctgaactttgatctgaaagaaattaacgatatttgcagcggctgccgcggccatgatggctttaccagcagctggcgcagcgtggcggataccctgcgccagaaagtggaagatgcggtgcgcgaacatccggattatcgcgtggtgtttaccggccatagcctgggcggcgcgctggcgaccgtggcgggcgcggatctgcgcggcaacggctatgatattgatgtgtttagctatggcgcgccgcgcgtgggcaaccgcgcgtttgcggaatttctgaccgtgcagaccggcggcaccctgtatcgcattacccataccaacgatattgtgccgcgcctgccgccgcgcgaatttggctatagccatagcagcccggaatattggattaaaagcggcaccctggtgccggtgacccgcaacgatattgtgaaaattgaaggcattgatgcgaccggcggcaacaaccagccgaacattccggatattccggcgcatctgtggtattttggcctgattggcacctgcctgtgacgcatgcgagctcggtaccc

SEQ ID NO: 12 - protein, 314 aa, Artificial Sequence
  the fusion-protein ompA-LipTl
MKKTAIAIAVALAGFATVAQAEFMRSSLVLFFVSAWTALASPIRREVSQDLFNQFNLFAQYSAAAYCGKNNDAPAGTNITCTGNACPEVEKADATFLYSFEDSGVGDVTGFLALDNTNKLIVLSFRGSRSIENWIGNLNFDLKEINDICSGCRGHDGFTSSWRSVADTLRQKVEDAVREHPDYRVVFTGHSLGGALATVAGADLRGNGYDIDVFSYGAPRVGNRAFAEFLTVQTGGTLYRITHTNDIVPRLPPREFGYSHSSPEYWIKSGTLVPVTRNDIVKIEGIDATGGNNQPNIPDIPAHLWYFGLIGTCL

SEQ ID NO: 13 - protein, 230 aa, Artificial Sequence
  AlkL mod1
VSFSNYKVIAMPVLVANFVLGAATAWANENYPAKSAGYNQGDWVASFNFSKVYVGEELGDLNVGGGALPNADVSIGNDTTLTFDIAYFVSSNIAVDFFVGVPARAKFQGEKSISSLGRVSEVDYGPAILSLQYHYDSFERLYPYVGVGVGRVLFFDKTDGALSSFDIKDKWAPAFQVGLRYDLGNSWMLNSDVRYIPFKTDVTGTLGPVPVSTKIEVDPFILSLGASYVF